(19) United States
(12) Patent Application Publication
(10) Pub. No.: US 2008/0114322 A1
(43) Pub. Date: May 15, 2008

(54) Body panel for an adjustable pant-like disposable undergarment and the undergarment itself

(75) Inventors: Suzanne Marie Schmoker, Oshkosh, WI (US); Jeffrey James Loritz, Appleton, WI (US); Stacy Nicole Markovich, Appleton, WI (US); Jeffery Michael Tabor, Appleton, WI (US)

Correspondence Address: KIMBERLY-CLARK WORLDWIDE, INC.;Catherine E. Wolf, 401 NORTH LAKE STREET, NEENAH, WI, 54956, US

(73) Assignee: Kimberly-Clark Worldwide, Inc.

(21) Appl. No.: 11/595,229

(22) Filed: Nov. 10, 2006

(51) Int. Cl. A61F 13/15

(52) U.S. Cl. 604/385.03; 604/387; 604/396; 604/385.01

(57) ABSTRACT

A body panel for an adjustable pant-like disposable absorbent undergarment is disclosed. The body panel includes first, second and third regions separated by first and second lines of weakness. A tear facilitation member is secured to one of the region. The tear facilitation member has at least one unattached portion of sufficient size to be grasped between a person's thumb and index finger. The body panel also has an attachment member being secured to one of the other regions and being removeably secured to the tear facilitation member. As the attachment member is opened, the unattached portion can be grasped and pulled in an opposite direction to break the respective line of weakness.

### BACKGROUND OF THE INVENTION

Pant-like disposable undergarments for absorbing human discharges can appear similar in size and shape to regular cloth underwear which is designed to be laundered and reused two or more times. A disposable absorbent undergarment is intended to be worn by persons, including infants, toddlers, or adults, and is designed for a single or temporary use and is meant to be disposed of after being used once instead of being laundered or dry cleaned for re-use. Some examples of disposable undergarments include infant diapers, training pants, adult incontinence garments, feminine pants, etc.

Some pant-like disposable absorbent undergarments manufactured today resemble regular cloth underwear in that they have a waist opening and a pair of leg openings. Such pant-like disposable absorbent undergarments can be pulled up around the torso of a wearer in a similar fashion as regular cloth underwear. Still other pant-like disposable absorbent undergarments have an open or flat configuration and are designed to be placed adjacent to a wearer's torso and then rely upon one or more attachment tabs or fasteners to secure the undergarment around the wearer's torso. This design is beneficial for bed bound users who may be immobile and who need assistance in securing the undergarment in place. Still other adjustable, pant-like absorbent undergarments contain attachment means for opening and closing the waist opening after the undergarment has been positioned around the wearer's torso. The adjustable undergarment has an advantage in that the wearer does not have to remove outer clothing in order to check the status of the undergarment or to remove the undergarment from their body. One adjustable, pant-like disposable absorbent undergarment currently being commercially sold by Kimberly-Clark Corporation uses a pair of lines of weakness. The lines of weakness extend from the waist opening to one of the leg openings. The lines of weakness are designed to be broken either prior to positioning the undergarment around the user's torso or while the undergarment is already positioned about the wearer's torso. A pair of attachment members is then utilized to refasten the undergarment so that it is snug about the wearer's torso.

It has been found that a major portion of each of the lines of weakness is visually hidden by the respective attachment member and some users cannot see them and thereby do not know that they are present. In addition, each line of weakness may be ergonomically hard to tear open by older adults, some of who may be suffering from arthritis.

Now a body panel for an adjustable, pant-like disposable absorbent undergarment has been invented, as well as the undergarment itself, that utilizes a pair of tear facilitation members to easily cause the first and second lines of weakness to be broken.

### SUMMARY OF THE INVENTION

Briefly, this invention relates to a body panel for an adjustable pant-like disposable absorbent undergarment and the undergarment itself. The body panel includes a waist edge, a pair of side edges, and a pair of leg openings. The body panel has a first region, a second region and a third region separated by first and second lines of weakness. Each of the first and second lines of weakness extends between the waist edge and one of said pair of leg openings. The first and third regions are aligned adjacent to one of the pair of side edges. A tear facilitation member is secured to one of the region. The tear facilitation member has at least one unattached portion of sufficient size to be grasped between a person's thumb and index finger. The body panel also has an attachment member being secured to one of the other regions and being removeably secured to the tear facilitation member. As the attachment member is opened, the unattached portion can be grasped and pulled in an opposite direction to break the respective line of weakness.

The adjustable pant-like disposable absorbent undergarment includes a front body panel, a back body panel and an absorbent assembly secured to the front and back body panels. The front and back body panels are joined together by a pair of seams to form a waist opening and a pair of leg openings. The front body panel contains the features identified above which facilitate inspection and possible removal of the undergarment from a wearer's body without requiring the wearer to remove any outer clothing.

### BRIEF DESCRIPTION OF THE DRAWINGS

FIG. 1 is a perspective view of an adjustable pant-like disposable absorbent undergarment having a front body panel with first and second lines of weakness, a tear facilitation member having a pair of unattached portions, and a pair of attachment members, wherein the pair of attachment members are shown being releasably attached over a second region of the front body panel.

FIG. 2 is a perspective view of the adjustable pant-like disposable absorbent undergarment shown in FIG. 1 when the pair of attachment members is released and the pair of unattached portions of the tear facilitation member is pulled back to expose the first and second lines of weakness.

FIG. 3 is a perspective view of the adjustable pant-like disposable absorbent undergarment shown in FIGS. 1 and 2 when the pair of attachment members and the pair of unattached portions of the tear facilitation member are pulled in opposite directions which causes the first and second lines of weakness to break.

FIG. 4 is a cross-sectional view of a portion of the front body panel shown in FIG. 1 taken along line 4-4 and showing a laminate structure with elastic strands sandwiched there between.

FIG. 5 is a perspective view of an alternative embodiment of an adjustable pant-like disposable absorbent undergarment having a front body panel with first and second lines of weakness, a pair of tear facilitation members each having an unattached portion, and a pair of attachment members, wherein the pair of attachment members are shown being releasably attached over a second region of the front body panel.

FIG. 6 is a perspective view of the adjustable pant-like disposable absorbent undergarment shown in FIG. 5 when the pair of attachment members is released and the unattached portion of each of the tear facilitation members are pulled back to expose the first and second lines of weakness.

FIG. 7 is a perspective view of the adjustable pant-like disposable absorbent undergarment shown in FIGS. 5 and 6 when the pair of attachment members and the unattached portion of each of the tear facilitation members are pulled in opposite directions which causes the first and second lines of weakness to break.

### DETAILED DESCRIPTION

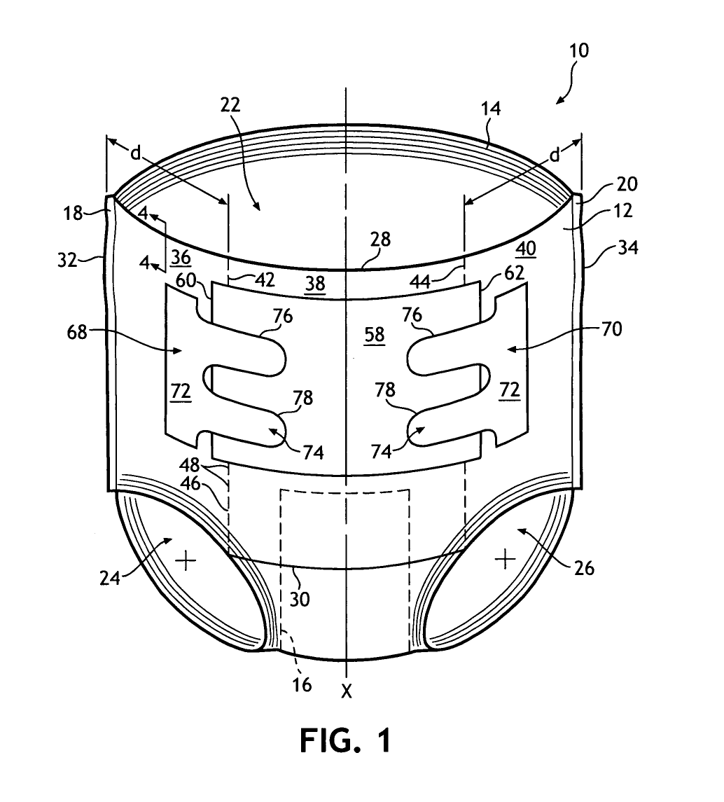

Referring to FIGS. 1-3, an adjustable pant-like disposable absorbent undergarment 10 having a longitudinal axis X-X is shown. The adjustable pant-like disposable absorbent undergarment 10 is designed to absorb liquid, semi-solid and/or solid waste discharged from a human being. A “disposable absorbent undergarment” as used herein is an article that is intended to be worn by persons, including infants, toddlers or adults, which is designed for a single or temporary use and is meant to be disposed of after being used once instead of being laundered or dry cleaned for re-use. The adjustable pant-like disposable absorbent undergarment 10 is designed to absorb and/or retain one or more bodily discharges of waste material such as urine, perspiration, excrement, feces, menses, menstrual fluid, as well as other liquid and/or solid waste.

The adjustable pant-like disposable absorbent undergarment 10 includes a front body panel 12, a back body panel 14 and an absorbent assembly 16 secured to the front and back body panels, 12 and 14 respectively. The front and back body panels, 12 and 14 respectively, are joined together by a pair of seams 18 and 20 to form a waist opening 22 and a pair of leg openings 24 and 26.

The front body panel 12 includes a waist edge 28, a crotch edge 30 and a pair of side edges 32 and 34. In a three piece construction wherein an absorbent assembly 16 is secured between the front body panel 12 and the back body panel 14, the crotch edge 30 is well defined. In absorbent undergarments of a different construction, the crotch edge 30 can be an imaginary line transversely drawn between the pair of leg openings 24 and 26 at a location where one considers the front body panel to end. The exact size and configuration of the front body panel 12 can vary to suit one's particular needs. The front body panel 12 has a first region 36, a second region 38 and a third region 40 separated by first and second lines of weakness, 42 and 44 respectively. The first region 36 is aligned adjacent to the side edge 32 and the third region 40 is aligned adjacent to the side edge 34. The second region 38 is located between the first and third regions, 36 and 40 respectively. As depicted, the second region 38 is centrally located and is bifurcated by the longitudinal axis X-X. However, the second region 38 could be offset such that it is aligned closer to one of the pair of side edges 32 or 34, if desired.

The first and second lines of weakness, 42 and 44 respectively, can be linear or non-linear in configuration. In FIGS. 1-3, the first and second lines of weakness, 42 and 44 respectively, are shown having a linear or straight configuration. The first and second lines of weakness, 42 and 44 respectively, extend longitudinally from approximate the waist opening 22 down to approximate one of the leg openings 24 or 26. The first and second lines of weakness, 42 and 44 respectively, can be aligned parallel to the longitudinal axis X-X or be angled thereto. For example, the first and second lines of weakness, 42 and 44 respectively, can be tapered relative to the longitudinal axis X-X, if desired.

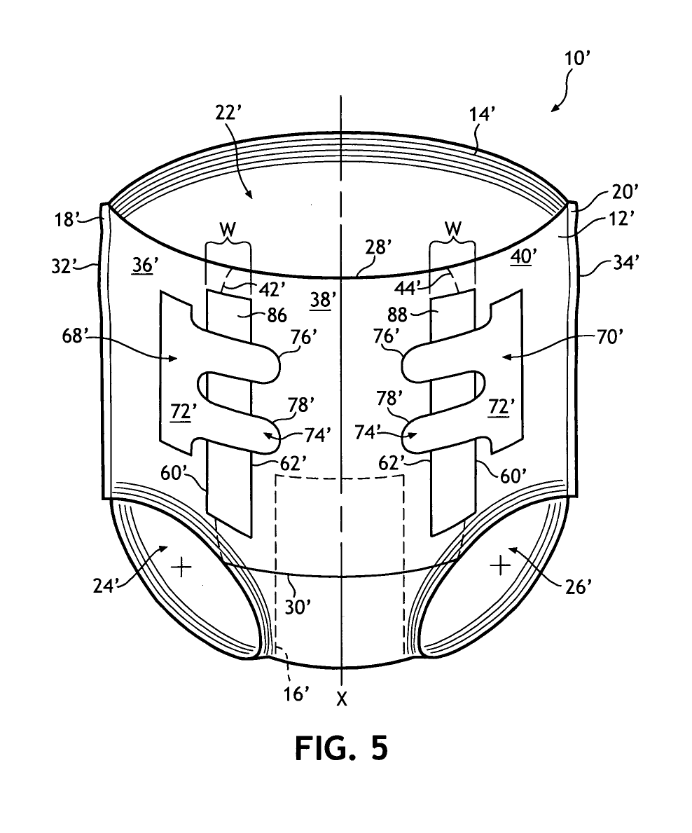

Referring to FIGS. 5-7, an alternative embodiment of an adjustable pant-like disposable absorbent undergarment 10′ is shown wherein the first and second lines of weakness, 42′ and 44′ respectively, are shown having a non-linear configuration. In FIGS. 5-7, the first and second lines of weakness, 42′ and 44′ respectively, have a curved or arcuate configuration and each is arranged in a concave relationship to one another. Alignment in a convex relationship is also possible. Sometimes, the use of curved or arcuate shaped first and second lines of weakness, 42′ and 44′ respectively, each aligned non-parallel to the longitudinal axis X-X, is desirable for it provides an aesthetically pleasing appearance to the undergarment 10′. Furthermore, the use of first and second lines of weakness, 42′ and 44′ respectively, each having a non-linear shape can be ergonomically easier to break open.

Returning to FIGS. 1-3, it should be noted that the first and second lines of weakness, 42 and 44 respectively, can have almost any imaginable shape or profile. Besides a straight line, each of the first and second lines of weakness, 42 and 44 respectively, could have a semi-circular shape, an arcuate shape, a concave shape, a convex shape, a V shape, a W shape, a zigzag pattern, a saw-tooth shape, a chevron shape, a sinusoidal shape, a geometrical shape having both linear and non-linear regions, etc. In addition, the first line of weakness 42 does not have to be a mirror image of the second line of weakness 44 with respect to the longitudinal axis X-X, although such an alignment is most desirable.

Still referring to FIGS. 1-3, each of the first and second lines of weakness, 42 and 44 respectively, extend from approximately the waist edge 28 of the front body panel 12 to one of the pair of leg openings 24 and 26. Another way of describing this is to say that the first and second lines of weakness, 42 and 44 respectively, extend from approximately the waist edge 28 of the front body panel 12 to approximately the crotch edge 30 of the front body panel 12. In some disposable absorbent undergarments, it may be desirable to have the first and second lines of weakness, 42 and 44 respectively, stop short of the leg openings 24 and 26 such that the material forming the front body panel 12 actually tears but does not follow a predetermined line of weakness.

In FIGS. 1-3, each of the first and second lines of weakness, 42 and 44 respectively, is shown as a straight perforated line consisting of multiple land areas 46 each separated by an open area 48. The length of each of the land areas 46 can be less than, equal to, or be greater than the length of each of the open areas 48. The ratio of the length of a land area 46 to the length of an adjacent open area 48 can be adjusted to increase or decrease the amount of force required to break the first and second lines of weakness, 42 and 44 respectively. The type of material into which the first and second lines of weakness, 42 and 44 respectively, are formed, the thickness of the material, the configuration of the first and second lines of weakness, 42 and 44 respectively, as well as other features, will all have an impact on the amount of force needed to break the first and second lines of weakness, 42 and 44 respectively. It should also be noted that the amount of force needed to start to break the first and second lines of weakness, 42 and 44 respectively, may be greater, equal to or less than the amount of force needed to continue to tear open the first and second lines of weakness, 42 and 44 respectively.

The first and second lines of weakness 42 and 44 can be formed such that each of the land areas 46 has a length that is equal to the length of each of the open areas 48. Alternatively, the length of the land and/or open areas, 46 and 48 respectively, can vary along a portion of or over the total length of the first and second lines of weakness 42 and 44. It has been found that when the length of the open areas 48 is greater than the length of the land areas 46, that the first and second lines of weakness, 42 and 44 respectively, can be easily broken. It is important to design the land and open areas, 46 and 48 respectively, such that the first and second lines of weakness, 42 and 44 respectively, are easy for the user to break yet ensure that the first and second lines of weakness, 42 and 44 respectively, will not break prematurely. Good results have been obtained by dimensioning the length of each of the open areas 48 to be at least two times greater than the length of each of the land areas 46. Desirably, the length of each of the open areas 48 will be at least three times greater than the length of each of the land areas 46. More desirably, the length of each of the open areas 48 will be at least four times greater than the length of each of the land areas 46.

Still referring to FIGS. 1-3, each of the first and second lines of weakness, 42 and 44 respectively, is shown being aligned parallel to one of the pair of seams 18 and 20. Such an arrangement provides for an aesthetically pleasing appearance to the front body panel 12. Each of the first and second lines of weakness, 42 and 44 respectively, extend downward from approximate the waist opening 22 to approximate one of the respective leg openings 24 and 26. The vertical distance to each of the first and second lines of weakness, 42 and 44 respectively, is located relative to the waist edge 28 and to the crotch edge 30 can vary. In addition, the horizontal distance to each of the first and second lines of weakness, 42 and 44 respectively, is located relative to the pair of seams 18 and 20 can also vary. A distance “d” is depicted in FIGS. 1-3 which represents the distance between each of the pair of seams 18 and 20 and the corresponding first and second lines of weakness, 42 and 44 respectively. One can increase the distance “d” by moving the first and second lines of weakness, 42 and 44 respectively, away from the pair of seams 18 and 20. When one increases the distance “d”, one may find that it is easier for the wearer of the disposable absorbent undergarment 10 to visually see and identify the first and second lines of weakness, 42 and 44 respectively, when he or she looks down at the front body panel 12. A manufacturer is free to vary the distance “d” to best suit the size and shape of a particular disposable absorbent undergarment 10.

As mentioned above, each of the first and second lines of weakness, 42 and 44 respectively, is depicted as a perforation line. However, each of the first and second lines of weakness, 42 and 44 respectively, can also be formed as a score line, a line having a reduced thickness relative to adjacent material, a line formed from a plurality of separation points, a line having breakable areas or zones, a line containing chain stitching, etc. Chain stitching is a stitch formed in a material such that when an end of the stitching is pulled, the stitch unravels and the material separates.

Furthermore, it is possible to utilize two or more perforation lines (not shown) for each of the first and second lines of weakness, 42 and 44 respectively. For example, each of the first and second lines of weakness, 42 and 44 respectively, can consist of a pair of perforation lines aligned parallel to one another and spaced from between about 1 millimeter (mm) to about 50 mm apart. Desirably, each of the pairs of perforation lines is spaced within 25 mm of one another. More desirably, each of the pairs of perforation lines is spaced within 15 mm of one another. Most desirably, each of the pairs of perforation lines is spaced within 10 mm of one another.

Referring now to FIG. 4, the front body panel 12 and/or the back body panel 14 can be formed from a single piece of material or they can be formed as a laminate consisting of two or more layers. The layers of the laminate can be of the same material or different material. In the cross-sectional view shown in FIG. 4, a laminate 50 is depicted formed from a first layer 52 and a second layer 54. Sandwiched between the first and second layers, 52 and 54 respectively, are two or more elastic strands 56. Desirably, from two to about a hundred elastic strands 56 can be utilized in either the front or back body panels, 12 and 14 respectively, depending upon the overall size of each panel. The elastic strands 56 can be formed from LYCRA. LYCRA is a registered trademark of E. I. Du Pont De Nemours & Co., having an office at 1007 Market Street, Wilmington, Del. 19898. The diameter and/or cross-sectional configuration of the elastic strands 56, the decitex (weight in grams per 10,000 meters) of the elastic strands 56, and the tension imparted into the elastic strands 56 can all be varied to suit one's particular product needs. The number of elastic strands present in the front body panel 12 can be less than, equal to or greater than the number of elastic strands present in the back body panel 14. The exact number of elastic strands 56 that are utilized should be sufficient to ensure that the disposable absorbent undergarment 10 snuggly conforms to the wearer's torso.

The elastic strands 56 can be coated with an adhesive, as is taught in U.S. Pat. Nos. 6,719,846; 6,863,225; 6,936,125 and 7,014,911. These patents are incorporated by reference and made a part hereof. By adhesively coating each of the elastic strands 56, instead of slot coating a major portion of the inner surface of at least one of the first and second layers, 52 and 54 respectively, softer front and back body panels, 12 and 14 respectively, can be obtained. Wearer's of disposable absorbent undergarments prefer a product that has a softer feel since it approaches the feel of cotton underwear which they may have worn for a number of years.

It should be noted that the front and/or back body panels, 12 and/or 14 respectively, can be formed from a breathable or a non-breathable material. Desirably, the front and back body panels, 12 and 14 respectively, are formed from a breathable material or a material that is treated or processed to be breathable. Spunbond and bonded carded webs are two breathable materials that work well as front and back body panels, 12 and 14 respectively, in disposable absorbent undergarments. Spunbond is a material manufactured and commercially sold by Kimberly-Clark Corporation, having an office at 401 North Lake Street, Neenah, Wis. 54956. Bonded carded webs are produced and commercially sold by a variety of vendors. Other materials that can be used to form the front and back body panels, 12 and 14 respectively, include woven and non-woven materials formed from natural or synthetic fibers; polyolefins, such as polypropylene or polyethylene; thermoplastic films; as well as other materials known to those skilled in the art. A metallocene polypropylene works very well since it has a soft feel and can be easily ultrasonically bonded to itself.

Returning again to FIGS. 1-3, the front body panel 12 has a tear facilitation member 58 secured to the second region 38. For example, the tear facilitation member 58 can be secured or permanently attached to the front body panel 12 by an adhesive, such as a hot or cold melt adhesive, by an ultrasonic bond, by heat, by pressure, by a combination of heat and pressure, by a mechanical bond, by a chemical bond, or by other means known to those skilled in the art. The tear facilitation member 58 has a first side edge 60 and an oppositely aligned second side edge 62. The first side edge 60 can extend over and beyond the first line of weakness 42 toward the side edge 32 and the second side edge 62 can extend over and beyond the second line of weakness 44 toward the side edge 34. Alternatively, the first side edge 60 can be aligned coterminuous with the first line of weakness 42 or be constructed to stop short of the first line of weakness 42. Likewise, the second side edge 62 can be aligned coterminuous with the second line of weakness 44 or be constructed to stop short of the second line of weakness 44.

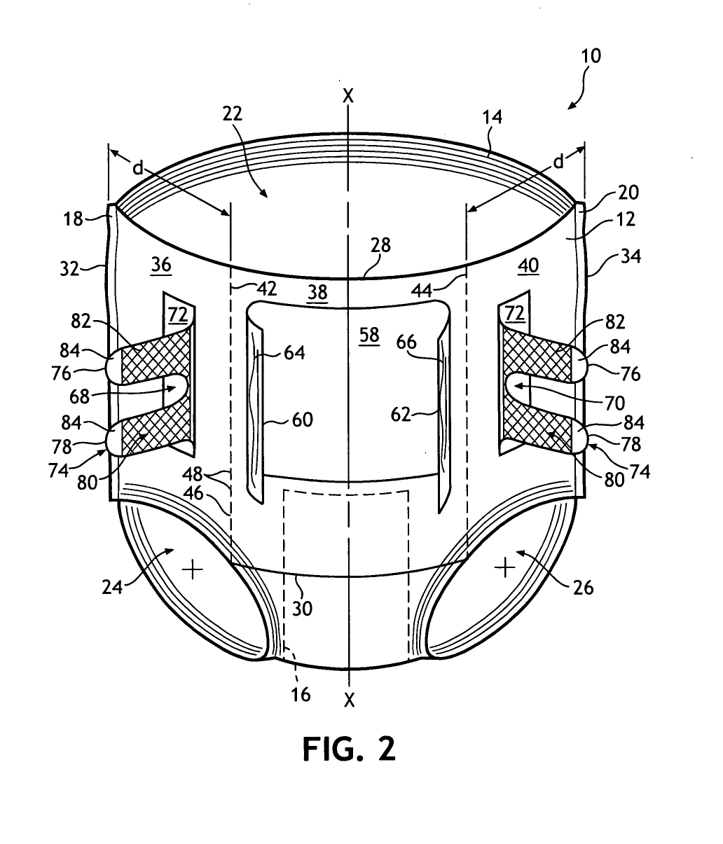

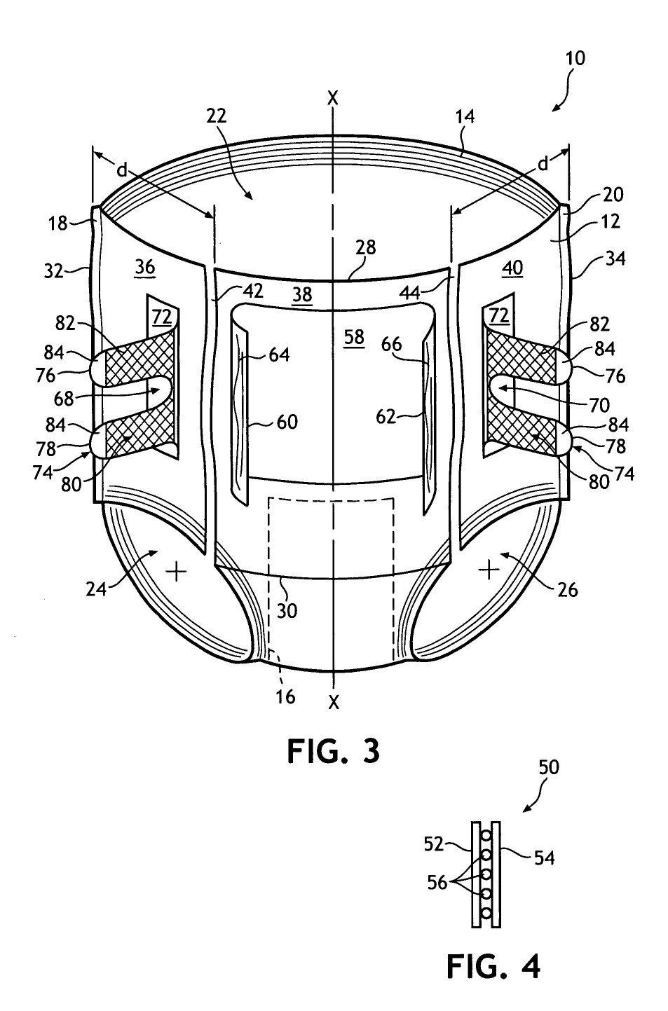

Turning now to FIGS. 2 and 3, the tear facilitation member 58 also has a pair of unattached portions 64 and 66, each of sufficient size to be grasped between a person's thumb and at least one finger, such as the index finger. The pair of unattached portions 64 and 66 is designed to be grasped between the person's thumb and index finger and can easily be pulled back towards the longitudinal central axis X-X. Each of the pair of unattached portions 64 and 66 overlaps a portion of the second region 38 and can cross one of the lines of weakness 42 or 44, if desired. Each of the pair of unattached portions 64 and 66 can also extend over a portion of either the first or third regions, 36 and 40, of the front body panel 12, if desired. The length and width of the tear facilitation member 58 can vary in dimension depending upon the size of the disposable absorbent undergarment 10 to which it is attached to. The shape of the tear facilitation member 58 can also vary to suit one's particular need.

Referring again to FIGS. 1-3, the front body panel 12 further includes a pair of attachment members 68 and 70. Each attachment member 68 and 70 includes a first portion 72 and a second portion 74. The first portion 72 of the attachment member 68 can be permanently secured to the first region 36 of the front body panel 12 and the first portion 72 of the other attachment member 70 can be permanently secured to the third region 40 of the front body panel 12. Alternatively, it should be evident to those skilled in the art that the first portion 72 of each of the attachment members 68 and 70 could be attached to the second region 38 and two tear facilitation members 58 could be employed with each being attached to one of the first and third regions, 36 and 40 respectively, if desired.

In the embodiment shown in FIGS. 1-3, the first portions 72 can be permanently attached using an adhesive, heat, pressure, a combination of heat and pressure, an ultrasonic bond, a mechanical fastener, a chemical bond or by other means known to those skilled in the art.

Each of the second portions 74 of the attachment members 68 and 70 are depicted as having a pair of flexible, outwardly extending finger-like tabs 76 and 78. It should be noted that a single tab of any desired size and shape could also be employed, as well as three or more tabs. The second portions 74 of the attachment member 68 can bridge across the first line of weakness 42 and the second portion 74 of the other attachment member 70 can bridge across the second line of weakness 44. Alternatively, the second portions 74 of the attachment members 68 and 70 can be coterminuous with or stop short of one of the lines of weakness, 42 or 44 respectively.

The second portions 74 of the attachment members 68 and 70 can be releasably attached to the second region 38 of the front body panel 12. Alternatively, the second portions 74 of the attachment members 68 and 70 can be releasably attached to another region of the front body panel 12.

Referring now to FIGS. 2 and 3, each of the second portions 74 of the attachment members 68 and 70 has an inner surface 80 that contains a fastener 82. The fastener 82 can be a mechanical fastener. Desirably the fastener 82 is a hook fastener. In FIGS. 2 and 3, the fastener 82 is shown as a plurality of fine hooks, such as VELCRO hooks. VELCRO is a trademark of Velcro USA, Inc., having an office at 406 Brown Avenue, Manchester, N.H. 03103. The hooks are designed to easily engage and be removed from a material wherein the material has a loose weave pattern or the fibers forming the material will allow the hooks to be attached to them. The mating material is commonly referred to as the loop member of a hook and loop fastener. The second region 38 of the front body panel 12 should be formed of such a material. When the hooks engage into the second region 38, a secure but releasable fastener is formed. The hooks can be easily removed from the loop material by pulling the finger-like tabs 76 and 78 outward away from the second region 38. Therefore, hook and loop fasteners are referred to as being releasable and can be fastened and released several times.

To assist the wearer of the disposable absorbent undergarment 10 or a caregiver to be able to easily release the finger-like tabs 76 and 78, the outer or terminal portions 84 of each tab 76 and 78 can be void of any fasteners 82. This feature allows one to easily grasp the nonattached terminal portions 84 and pull the finger-like tabs 76 and 78 away from the loop material into which the hooks are engaged.

It should be noted that one could reverse the hook and loop arrangement such that the loops are formed on the finger-like tabs 76 and 78 and the hooks are present on the second region 38, if desired.

Referring again to FIGS. 1-3, one can see that FIG. 1 depicts the pair of attachment members 68 and 70 being securely fastened to the second region 38 of the front body panel 12. If the wearer of the disposable absorbent undergarment 10 or a caregiver wishes to inspect the undergarment 10, he or she would open the pair of attachment members 68 and 70 to the position shown in FIG. 2. At this time, the unattached portions 64 and 66 of the tear facilitation member 58 are grasped and sequentially or simultaneously pulled in an opposite direction to break the first and second lines of weakness, 42 and 44 respectively. With the first and second lines of weakness, 42 and 44 respectively, being broken, see FIG. 3, the second region 38 of the disposable absorbent undergarment 10 can be moved outward away from the wearer's torso. The wearer can then inspect the absorbent assembly 16 to see if it needs to be changed. If so, the disposable absorbent undergarment 10 can be removed from about the wearer's torso and be replaced by another undergarment. If the absorbent assembly 16 is still capable of accepting additional body fluid, the second region 38 is moved back against the wearer's torso and the pair of attachment members 68 and 70 is refastened to the tear facilitation member 58.

Turning now to FIGS. 5-7, an alternative embodiment of a disposable absorbent undergarment 10′ is shown. For ease of understanding, numerals identical to those used to denote similar structural features of the disposable absorbent undergarment 10 are used, except the numerals are primed. The disposable absorbent undergarment 10′ differs from the first embodiment 10 in a number of ways. First, each of the first and second lines of weakness, 42′ and 44′ respectively, has a non-linear configuration. As shown, each of the first and second lines of weakness, 42′ and 44′ respectively, has a curved or arcuate shape. In addition, the first and second lines of weakness, 42′ and 44′ respectively, are arranged concave relative to one another and are arranged convex relative to the seams, 18′ and 20′ respectively. By “convex” it is meant that each of the first and second lines of weakness, 42′ and 44′ respectively, curve or bulge outward, as the exterior of a sphere, toward one of the adjacent seams 18′ or 20′.

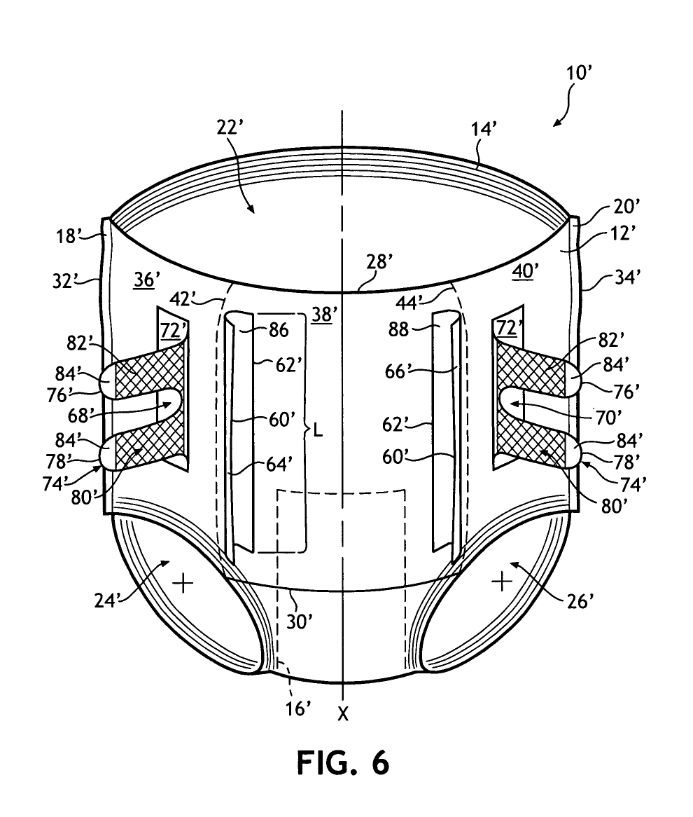

It should be noted that the radius of the curved first and second lines of weakness, 42′ and 44′ respectively, can vary. The exact radius will be partly dictated by the distance between the waist opening 22′ and the respective leg opening 24′ or 26′. As shown in FIG. 6, the curved first and second lines of weakness, 42′ and 44′ respectively, form mirror images of one another relative to the longitudinal central axis X-X.

Another difference exhibited in the disposable absorbent undergarment 10′ is that a pair of tear facilitation members 86 and 88 is present. Each of the pair of tear facilitation members 86 and 88 is secured to the second region 38′ of the front body panel 12′. For example, each of the pair of tear facilitation members 86 and 88 can be secured or permanently attached to the front body panel 12′ by an adhesive, such as a hot or cold melt adhesive, by an ultrasonic bond, by heat, by pressure, by a combination of heat and pressure, by a mechanical bond, by a chemical bond, or by other means known to those skilled in the art. As stated above, one could secure the pair of tear facilitation members 86 and 88 to the first and third regions 36′ and 40′ respectively, if desired.

Each of the pair of tear facilitation members 86 and 88 has a first side edge 60′ and an oppositely aligned second side edge 62′. The first side edge 60′ of the tear facilitation member 86 can extend over and beyond the first line of weakness 42′ toward the side edge 32′. Alternatively, the first side edge 60′ of the tear facilitation member 86 could be aligned coterminuous with the first line of weakness 42′ or be constructed to stop short of the first line of weakness 42′. Likewise, the first side edge 60′ of the tear facilitation member 88 can extend over and beyond the second line of weakness 44′ toward the side edge 34′. Alternatively, the first side edge 60′ of the tear facilitation member 88 could be aligned coterminuous with the second line of weakness 44′ or be constructed to stop short of the second line of weakness 44′.

Referring again to FIG. 5, each one of the pair of tear facilitation members 86 and 88 can have a width “w” which is of a sufficient dimension to cover the respective first or second line of weakness 42′ and 44′, if desired. The width “w” dimension of each of the pair of tear facilitation members 86 and 88 is desirably from between about 0.25 inches (about 0.62 cm) to about 5 inches (about 12.5 cm). More desirably, the width “w” dimension of each of the pair of tear facilitation members 86 and 88 is from between about 0.5 inches (about 1.25 cm) to about 3 inches (about 7.6 cm). Even more desirably, the width “w” dimension of each of the pair of tear facilitation members 86 and 88 is from between about 1 inch (about 2.5 cm) to about 2 inches (about 5 cm).

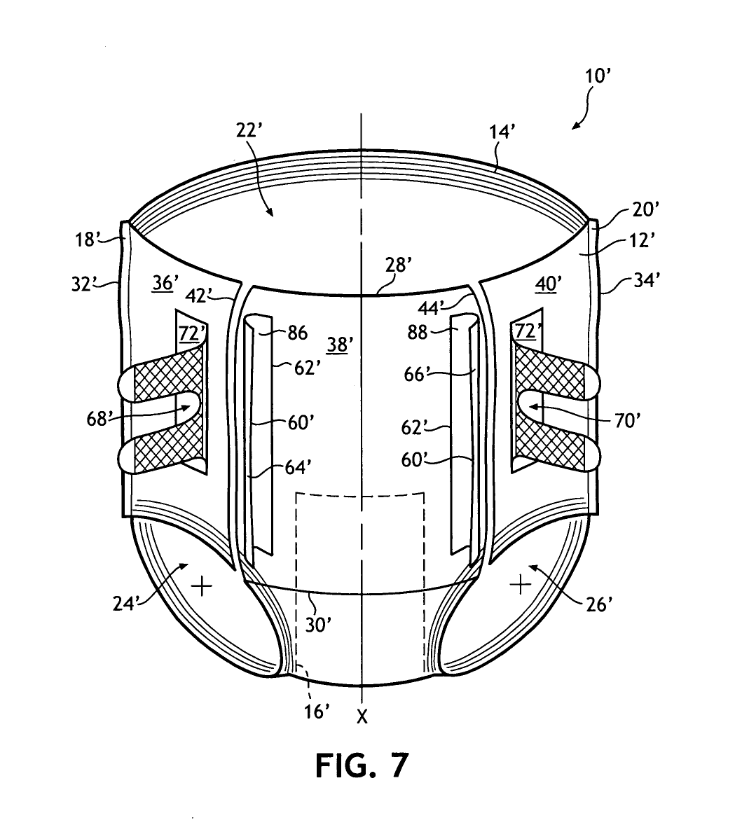

Returning now to FIGS. 6 and 7, each of the pair of tear facilitation members 86 and 88 also has an unattached portion, 64′ and 66′ respectively. Each unattached portion 64′ and 66′ is of sufficient size to be grasped between a person's thumb and at least one finger, such as the index finger. The unattached portions 64′ and 66′ are designed to be grasped between the person's thumb and index finger and can easily be pulled back towards the longitudinal central axis X-X. Each of the unattached portions 64′ and 66′ can overlap a portion of the second region 38′. Each of the unattached portions 64′ and 66′ can cross one of the lines of weakness 42′ or 44′ and extends over a portion of either the first or third regions, 36′ and 40′, of the front body panel 12′. Alternatively, each of the unattached portions 64′ and 66′ can be aligned even with or stop short of one of the lines of weakness 42′ or 44′. In this alternative configuration, each of the unattached portions 64′ and 66′ would not extend over a portion of either the first or third regions, 36′ and 40′, of the front body panel 12′.

The length and width of the pair of tear facilitation members 86 and 88 can vary in dimension depending upon the size of the disposable absorbent undergarment 10′ to which it is attached to. The shape of each of the pair of tear facilitation members 86 and 88 can also vary to suit one's particular product design. For example, each of the pair of tear facilitation members 86 and 88 can have a length “I” that is less than the distance between the waist edge 28′ and one of the pair of leg openings 24′ or 26′, see FIG. 6. Desirably, each of the pair of tear facilitation members 86 and 88 will have a length “I” that extends over a major portion of the distance located between the waist edge 28′ and one of the pair of leg openings 24′ or 26′.

Still referring to FIGS. 6 and 7, the disposable absorbent undergarment 10′ also includes a pair of attachment members 68′ and 70′ that function in a similar fashion to those described above in relation to the undergarment 10. Each of the pair of attachment members 68′ and 70′ has a first portion 72′ and a second portion 74′. The first portion 72′ of the attachment member 68′ is permanently secured to the first region 36′ of the front body panel 12′ and the first portion 72′ of the other attachment member 70′ is permanently secured to the third region 40′ of the front body panel 12′. Alternatively, one skilled in the art will recognize that the first portion 72′ of the attachment member 68′ and the first portion 72′ of the other attachment member 70′ can be permanently secured to the second region 38′ of the front body panel 12′. The first portions 72′ can be permanently attached using an adhesive, heat, pressure, a combination of heat and pressure, an ultrasonic bond, a mechanical fastener, a chemical bond or by other means known to those skilled in the art.

Each of the second portions 74′ of the attachment members 68′ and 70′ are depicted as having a pair of flexible, outwardly extending finger-like tabs 76′ and 78′. It should be noted that a single tab of any desired size and shape could also be employed, as well as three or more tabs. The second portions 74′ of the attachment member 68′ can bridge across the first line of weakness 42′ and the second portion 74′ of the other attachment member 70′ can bridge across the second line of weakness 44′. Alternatively, second portions 74′ of the attachment member 68′ and 70′ can be aligned even with or stop short of the first and second lines of weakness, 42′ and 44′ respectively. The second portions 74′ can be releasably attached to the second region 38′ of the front body panel 12′. Alternatively, the second portions 74′ can be releasably attached to another region of the front body panel 12′.

Each of the second portions 74′ of the attachment members 68′ and 70′ has an inner surface 80′ that contains a fastener 82′. The fastener 82′ can be a mechanical fastener. Desirably, the fastener 82′ is a hook fastener. In FIGS. 6 and 7, the fastener 82′ is shown as a plurality of fine hooks, such as VELCRO hooks. The hooks are designed to easily engage and be removed from a material wherein the material has a loose weave pattern or the fibers forming the material will allow the hooks to be attached to them. The mating material is commonly referred to as the loop member of a hook and loop fastener. The second region 38′ of the front body panel 12′ should be formed of such a material. When the fasteners 82′ engage into the second region 38′, a secure but releasable bond is formed. The fasteners 82′ can be easily removed from the loop material by pulling the finger-like tabs 76′ and 78′ outward away from the second region 38′. Therefore, hook and loop fasteners are referred to as being releasable and can be fastened and released multiple times.

To assist the wearer of the disposable absorbent undergarment 10′ or a caregiver to be able to easily release the finger-like tabs 76′ and 78′, the outer or terminal portions 84′ of each tab 76′ and 78′ can be void of any fasteners 82′. This feature allows one to easily grasp the nonattached terminal portions 84′ and pull the finger-like tabs 76′ and 78′ away from the loop material into which the hooks are engaged.

It should be noted that one could reverse the hook and loop arrangement such that the loops are formed on the finger-like tabs 76′ and 78′ and the hooks are present on the second region 38′, if desired.

Referring again to FIGS. 5-7, one can see that FIG. 5 depicts the pair of attachment members 68′ and 70′ being securely fastened to the second region 38′ of the front body panel 12′. If the wearer of the disposable absorbent undergarment 10′ or a caregiver wishes to inspect the undergarment 10′, he or she would open the pair of attachment members 68′ and 70′ to the position shown in FIG. 6. At this time, the unattached portions 64′ and 66′ of the tear facilitation member 86 and 88 are grasped and sequentially or simultaneously pulled in an opposite direction to break the first and second lines of weakness, 42′ and 44′ respectively. With the first and second lines of weakness, 42′ and 44′ respectively, being broken, see FIG. 7, the second region 38′ of the disposable absorbent undergarment 10′ can be moved outward away from the wearer's torso. The wearer can then inspect the absorbent assembly 16′ to see if it needs to be changed. If so, the disposable absorbent undergarment 10′ can be removed from about the wearer's torso and be replaced by another undergarment. If the absorbent assembly 16′ is still capable of accepting additional body fluid, the second region 38′ is moved back against the wearer's torso and the pair of attachment members 68′ and 70′ is refastened to the pair of tear facilitation member 86 and 88.

It should be noted that each of the pair of tear facilitation members 86 and 88 can be constructed of a material having a color that is different from the material forming the second region 38′. For example, for a disposable absorbent undergarment 10′ which is white in color, the pair of tear facilitation members 86 and 88 can be pink, blue, green, red, etc. in color. It should be readily recognized by those skilled in the art that any combination of colors or contrasting color tones can be utilized. A dark color relative to a light color of the same tint or hue can also be used. One could even use glow in the dark colors for the pair of tear facilitation members 86 and 88, if desired. In addition, each of the pair of tear facilitation members 86 and 88 can be constructed of a material having a color that is different from the material forming the pair of attachment members 68′ and 70′. Again, if the pair of attachment members 68′ and 70′ is white in color, the pair of tear facilitation members 86 and 88 can be of some different color.

The pair of tear facilitation members 86 and 88 can also be constructed of a material having a different tactile property and/or having a different tactile pattern from the material forming the second region 38′. For example, the pair of tear facilitation members 86 and 88 can be constructed from a material that is coarser to the touch and/or which contains a tactile pattern that visually distinguishes it from the second region 38′.

Still further, each of the pair of tear facilitation members 86 and 88 can be highlighted to be visually distinguishable from the front body panel 12′. Highlighting can be accomplished by printing a color, pattern or image onto the pair of tear facilitation members 86 and 88. Other means known by those skilled in the art for highlighting the pair of tear facilitation members 86 and 88 can also be used. Some alternatives to highlighting the pair of tear facilitation members 86 and 88 can include dying, embossing, painting, spraying, chemically treating, etc.

It should also be recognized that color adhesives can be employed in permanently securing the tear facilitation member 58 to the second region 38 of the front body panel 12 or for securing the tear facilitation members 86 and 88 to the second region 38′ of the front body panels 12′. Color adhesives can also be used to permanently secure the pair of attachment members 68 and 70 to the first and third regions, 36 and 40 respectively, or for securing the pair of attachment members 68′ and 70′ to the first and third regions 38′ and 40′ respectively. The color adhesives can serve the same function as using a different color material to visually distinguish where the first and second lines of weakness, 42 and 44 or 42′ and 44′, are located. The wearer of the disposable absorbent undergarment 10 or 10′ will be able to notice the first and second lines of weakness, 42 and 44 or 42′ and 44′, which are located between the color adhesives.

While the invention has been described in conjunction with two specific embodiments, it is to be understood that many alternatives, modifications and variations will be apparent to those skilled in the art in light of the aforegoing description. Accordingly, this invention is intended to embrace all such alternatives, modifications and variations that fall within the spirit and scope of the appended claims.

We claim:

1. A body panel for an adjustable pant-like disposable absorbent undergarment comprising:
a) a waist edge, a pair of side edges and a pair of leg openings, said body panel having a first region, a second region and a third region separated by first and second lines of weakness, each of said first and second lines of weakness extending between said waist edge and one of said leg openings, and said first and third regions being aligned adjacent to one of said pair of side edges;
b) a tear facilitation member secured to one of said regions and having at least one unattached portion of sufficient size to be grasped between a person's thumb and index finger; and
c) an attachment member being secured to one of said regions said tear facilitation member is not secured to and being removeably secured to said tear facilitation member, whereby as said attachment member is opened, said respective unattached portion of said tear facilitation member can be grasped and pulled in an opposite direction to break said respective line of weakness.

2. The body panel of claim 1 wherein said tear facilitation member is secured to said second region and extends over said first and second lines of weakness.

3. The body panel of claim 1 wherein said attachment member bridges across one of said first and second lines of weakness.

4. The body panel of claim 1 wherein said tear facilitation member has a length that extends over a major portion of the distance located between said waist edge and said pair of leg openings.

5. The body panel of claim 1 wherein said tear facilitation member has a length that is less than the distance located between said waist edge and one of said pair of leg openings.

6. The body panel of claim 1 wherein a pair of attachment members are present and each contains a fastener which can be releasably attached to said tear facilitation member.

7. The body panel of claim 6 wherein said fastener is a mechanical fastener.

8. The body panel of claim 7 wherein said fastener is a hook fastener.

9. The body panel of claim 1 wherein said second region functions as a loop fastener.

10. A body panel for an adjustable pant-like disposable absorbent undergarment comprising:
a) a waist edge, a pair of side edges and a pair of leg openings, said body panel having a first region, a second region and a third region separated by first and second lines of weakness, each of said first and second lines of weakness extending between said waist edge and one of said pair of leg openings, and said first and third regions being aligned adjacent to one of said pair of side edges;
b) a pair of tear facilitation members each secured to said one of said regions, and each of said pair of tear facilitation members having an unattached portion of sufficient size to be grasped between a person's thumb and index finger; and
c) a pair of attachment members each being secured to a region of said body panel to which said pair of tear facilitation members are not secured, each of said pair of attachment members being releasably attached to one of said pair of tear facilitation members, whereby as each of said pair of attachment members is opened, said unattached portion of said respective tear facilitation member can be grasped and pulled in an opposite direction to break said respective line of weakness.

11. The body panel of claim 10 wherein each of said tear facilitation members has a width of from between about 0.25 inches to about 5 inches.

12. The body panel of claim 10 wherein each of said first and second lines of weakness has a non-linear configuration and each of said tear facilitation members has a sufficient dimension to cover said respective line of weakness.

13. The body panel of claim 10 wherein each of said tear facilitation members is constructed of a material having a color that is different from said second region.

14. The body panel of claim 10 wherein each of said tear facilitation members is constructed of a material having a color that is different from said pair of attachment members.

15. The body panel of claim 10 wherein each of said tear facilitation members is highlighted to be visually distinguishable from said body panel.

16. An adjustable pant-like disposable absorbent undergarment comprising:
a) a front body panel;
b) a back body panel;
c) an absorbent assembly secured to said front and back body panels, and said front and back body panels being joined together by a pair of seams to form a waist opening and a pair of leg openings;
d) first and second lines of weakness extending between said waist opening to one of said leg openings, said first and second lines of weakness separating said front body panel into a first region, a second region and a third region, and said first and third regions being aligned adjacent to one of said pair of seams;
e) a pair of tear facilitation members each secured to said second region and each extending over one of said pair of lines of weakness, and each of said tear facilitation members having an unattached portion of sufficient size to be grasped between a person's thumb and index finger; and
f) a pair of attachment members each being secured to one of said first and third regions, each of said pair of attachment members bridging across one of said pair of lines of weakness and being releasably attached to said second region, whereby as each of said pair of attachment members is opened, said unattached portion of said respective tear facilitation member can be grasped and pulled in an opposite direction to break said respective line of weakness.

17. The adjustable pant-like disposable undergarment of claim 16 wherein each of said tear facilitation members is constructed of a material having a color that is different from said second region.

18. The adjustable pant-like disposable undergarment of claim 16 wherein each of said tear facilitation members is constructed of a material having a color that is different from said pair of attachment members.

19. The adjustable pant-like disposable undergarment of claim 16 wherein each of said pair of tear facilitation members has a width of from between about 0.5 inches to about 3 inches.

20. The adjustable pant-like disposable undergarment of claim 16 wherein each of said tear facilitation members is secured to said front body panel by colored adhesives.
